(19) United States
(12) Patent Application Publication
(10) Pub. No.: US 2026/0090901 A1
(43) Pub. Date: Apr. 2, 2026

(54) Neurovascular Stent Delivery Apparatus

(71) Applicant: ACCUMEDICAL BEIJING LTD., Beijing (CN)

(72) Inventors: Tai Dac Tieu, Fountain Valley, CA (US); Zihao Luo, Costa Mesa, CA (US); Kirk Wu, Costa Mesa, CA (US)

(73) Assignee: ACCUMEDICAL BEIJING LTD., Beijing (CN)

(21) Appl. No.: 18/903,378

(22) Filed: Oct. 1, 2024

(51) Int. Cl. A61F 2/966; A61F 2/95

(52) U.S. Cl. CPC A61F 2/966; A61F 2002/9505; A61F 2002/9665

(57) ABSTRACT

A stent delivery apparatus is disclosed and configured for selectively positioning a self-expanding stent relative to a target site. In at least one embodiment, a delivery mechanism is configured for being selectively positionable coaxially within and removably engageable with the stent. The delivery mechanism provides at least one self-expanding delivery sleeve, with each delivery sleeve providing a proximal end cap positioned at a proximal end of said delivery sleeve and a distal end cap positioned at an opposing distal end of said delivery sleeve. An engagement mechanism is positioned coaxially within said delivery sleeve and provides an elongate engagement shaft engaged with the proximal end cap and extending a distance toward the distal end cap, and an at least one stent catch positioned substantially on a distal end of the engagement shaft and configured for removable engagement with the stent.

### RELATED APPLICATIONS

Not applicable.

### BACKGROUND

The subject of this patent application relates generally to intravascular therapeutic devices and delivery systems, and more particularly to a neurovascular stent delivery apparatus capable of more easily positioning and repositioning a stent prior to complete deployment.

Applicant hereby incorporates herein by reference any and all patents and published patent applications cited or referred to in this application.

By way of background, blood vessel disorders, specifically those affecting the neurovasculature, including intracranial atherosclerotic disease (ICAD) and aneurysms are a significant point of interest for innovation. Per the Center for Disease Control and Prevention (CDC), in 2020, cerebrovascular diseases were the fifth highest cause of deaths of any cause in the United States.

Stenting involves entering a patient's vascular system and deploying a mesh tube to provide structural support to a blood vessel. This may be done to treat blood vessels that have fatty deposits or plaque built up in the blood vessel, otherwise known as atherosclerosis, or to support a vessel that was damaged or whose structural integrity is in question. In the neurovasculature, stenting is most notably used to prevent strokes or treat recurring strokes.

Stents may either be self-expanding or expanded via a mechanical method such as a balloon. As manufactured, stents are loaded onto an appropriate delivery system, either inside of an introducer sheath for a self-expanding stent, or onto a balloon catheter for a non-self-expanding stent. Procedurally, the interventionalist will locate the treatment site using a combination of accessories, typically including introducers, guide catheters, guide wires, microcatheters, and fluoroscopic imaging techniques. The interventionalist will then insert the delivery system into a guide catheter or microcatheter appropriately sized for the anatomy being treated. The delivery system with the pre-loaded stent will then be advanced to the target location and deployed by either retracting the guide catheter or microcatheter for self-expanding stents or by applying internal pressure to a balloon catheter mechanically forcing the stent to open for a non-self-expanding stent.

Stenting in small and tortuous vessels, such as the neurovasculature, introduces additional challenges due to the tortuosity of the anatomy and the reduced size of the vessels. The increased tortuosity of the neurovasculature requires that devices used be more flexible, and the reduced size of the vessels requires that devices and any accessories reduce their size to accommodate the smaller vessels. Due to the small and tortuous vessels of the neurovasculature, devices intended to be used in this part of the anatomy must be specially designed to function appropriately. Special considerations must also be given to the limited use of accessory devices as access of multiple devices simultaneously in the neurovascular may be difficult or impossible.

When stents are being delivered, accuracy of stent placement is critical to ensure that the stent is as effective as possible. In more tortuous anatomies, such as near bifurcations or aneurysms, the accuracy of stent placement is of paramount importance. Should the stent be placed inaccurately, such that it extends beyond the intended vessel wall and into the open vessel, there is significant patient risk for thrombus formation. This risk increases in critical anatomies, such as the neurovasculature, where an improperly placed stent may increase the risk of stroke in the patient. In larger vessels, there are established techniques using accessory devices, such as additional guidewires or balloon catheters, which can improve the accuracy of stent placement. In the neurovasculature, the reduced vessel diameter and tortuosity either prevents the usage of these techniques or increases their risk and/or difficulty. Therefore, there is a distinct need for a stent and stent delivery system which would improve the accuracy of stent placement without the need for accessories or advanced techniques.

One method of providing additional stent placement accuracy is to allow the stent to be recaptured prior to complete deployment. This would provide the interventionalist an opportunity to reposition the stent if it does not deploy in the desired location. An example of such a prior art solution can be found in US 2023/0338175 to Park et al., wherein the stent includes a cylindrical portion forming a stent frame having an outer lattice network of a plurality of interconnecting segments configured to exert a radial force against an inner wall of a blood vessel, and a corresponding stent delivery system includes a locking mechanism configured to engage with the proximal end of the stent frame when covered in a sheath (such as a catheter) along with a push wire. The locking mechanism includes a proximal retention disk and a distal retention disk, the distal retention disk having a plurality of slots configured to engage with an equal plurality of struts formed on a proximal end of the stent. Accordingly, the laterally opposing tines of each slot protrude through apertures in the latticed stent so as to assist in the movement of the stent through the sheath. However, in such prior art solutions, the stent delivery system often leaves the tines exposed once the stent expands and detaches from the delivery system. As a result, there is a chance that one or more of the tines may remain in protruding engagement with an aperture of the stent (particularly when the stent is positioned within a curved vessel), such that subsequent removal of the detached stent delivery system could catch on the stent and cause it to be pulled out of position and lead to procedural complications.

Thus, there still remains a need for a stent and corresponding delivery system which are able to provide a repositionable, low-profile stent which can meet the functional requirements to effectively treat blood vessel disorders without sacrificing patient outcomes or introducing additional risk. Aspects of the present invention fulfill these needs and provide further related advantages as described in the following summary.

It should be noted that the above background description includes information that may be useful in understanding aspects of the present invention. It is not an admission that any of the information provided herein is prior art or relevant to the presently claimed invention, or that any publication specifically or implicitly referenced is prior art.

### SUMMARY

Aspects of the present invention teach certain benefits in construction and use which give rise to the exemplary advantages described below.

The present invention solves the problems described above by providing a neurovascular stent apparatus configured for selectively positioning a self-expanding stent relative to a target site, the stent being constructed out of a plurality of individual stent struts braided together so as to define plurality of stent gaps between the intersecting stent struts along a length of the stent or out of a laser cut tube forming a frame having an outer lattice network of a plurality of interconnecting stent struts with stent gaps between the stent struts and the stent configured for moving between one of an elongated undeployed shape—wherein an outer stent diameter of the stent is smaller than an inner catheter diameter of a catheter—and a relatively larger deployed shape—wherein the outer stent diameter is sized and configured for allowing the stent to effectively fill the target site. In at least one embodiment, a delivery mechanism is configured for being selectively positionable coaxially within and removably engageable with the stent. The delivery mechanism provides a proximal push wire engaged with a proximal end of the delivery mechanism and configured for moving each of the delivery mechanism and the stent through the catheter, a distal push wire positioned at an opposing distal end of the delivery mechanism and configured for assisting in guiding each of the delivery mechanism and the stent through the catheter, and an at least one self-expanding delivery sleeve positioned between the proximal push wire and the distal push wire. Each of the at least one delivery sleeve provides a plurality of individual sleeve strands braided together so as to define plurality of sleeve gaps between the intersecting sleeve strands along a length of said delivery sleeve, said delivery sleeve configured for moving between one of an elongated undeployed shape—wherein an outer sleeve diameter of said delivery sleeve is smaller than an inner stent diameter of the stent for allowing said delivery sleeve to be coaxially positioned within the stent when the stent is in its undeployed shape—and a relatively larger deployed shape. A proximal end cap is positioned at a proximal end of said delivery sleeve for maintaining the braided arrangement of the sleeve strands of said delivery sleeve, and a distal end cap is positioned at an opposing distal end of said delivery sleeve for maintaining the braided arrangement of the sleeve strands of said delivery sleeve. An engagement mechanism is positioned coaxially within said delivery sleeve and provides an elongate engagement shaft engaged with the proximal end cap and extending a distance toward the distal end cap, and an at least one stent catch positioned substantially on a distal end of the engagement shaft and configured for removable engagement with the stent when said delivery sleeve and the stent are each in their undeployed shapes, the at least one stent catch having a length that is less than a radius of said delivery sleeve when said delivery sleeve is in its deployed shape, such that the at least one stent catch becomes disengaged from said delivery sleeve when said delivery sleeve and the stent are each in their undeployed shapes. During use of the apparatus, with the at least one delivery sleeve positioned within the stent and the stent positioned within the catheter in their respective undeployed shapes, upon the catheter reaching the target site, the proximal push wire is used to push the delivery mechanism which, in turn, advances the at least one delivery sleeve and stent via the at least one stent catch through the catheter, and upon each of the at least one delivery sleeve and stent exiting the catheter, each of the at least one delivery sleeve and stent automatically change into their respective deployed shapes, thereby causing the at least one stent catch to become disengaged from both the at least one delivery sleeve and the stent.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIGS. 1 and 2 are partial side elevational views of an exemplary neurovascular stent delivery apparatus, in accordance with at least one embodiment;

FIGS. 3 and 4 are partial side elevational views of an exemplary delivery mechanism of the apparatus, in accordance with at least one embodiment;

FIG. 5 is a partial perspective view of an exemplary engagement mechanism of the delivery mechanism, in accordance with at least one embodiment;

FIG. 6 is a front elevational view thereof, in accordance with at least one embodiment;

FIG. 7 is a partial perspective view of the delivery mechanism, in accordance with at least one embodiment;

FIG. 8 is a partial perspective view of the apparatus, in accordance with at least one embodiment;

FIG. 9 is a partial top plan view of the apparatus, in accordance with at least one embodiment;

FIGS. 10 and 11 are partial side elevational views of a further exemplary neurovascular stent delivery apparatus, in accordance with at least one embodiment;

FIG. 12 is a further partial side elevational view thereof, with a stent omitted for clarity, in accordance with at least one embodiment;

FIG. 13 is a further partial side elevational view of the apparatus of FIG. 10, in accordance with at least one embodiment;

FIG. 14 is a perspective view of the apparatus of FIG. 10, in accordance with at least one embodiment;

FIG. 15 is a partial perspective view of a further exemplary embodiment of the apparatus, in accordance with at least one embodiment;

FIG. 16 is a partial side elevational view of a further exemplary delivery mechanism of the apparatus, in accordance with at least one embodiment; and

FIG. 17 is a partial side elevational view of a still further exemplary delivery mechanism of the apparatus, in accordance with at least one embodiment.

The above described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

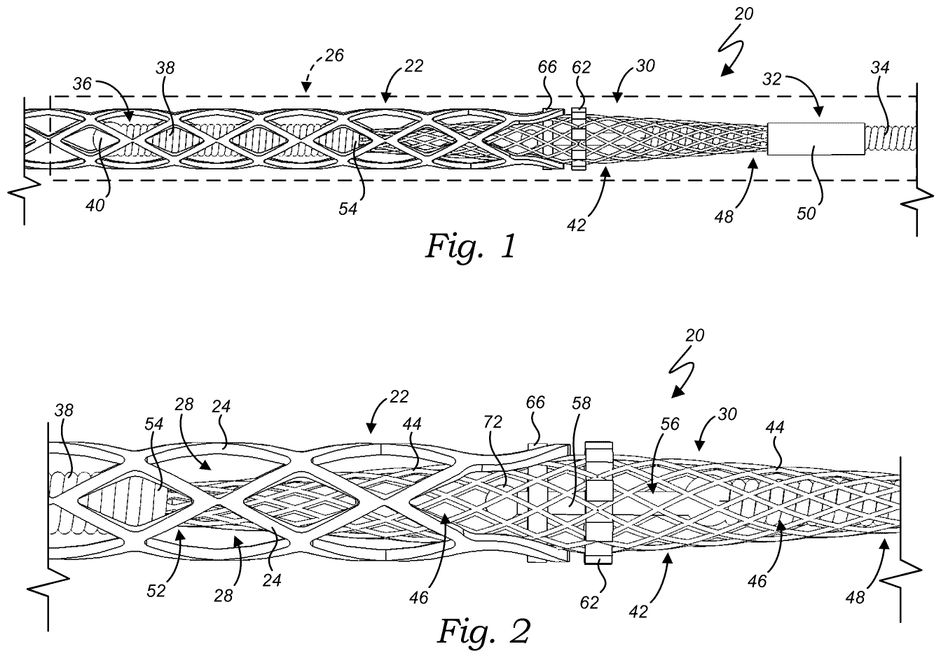

Turning now to FIGS. 1 and 2, there are shown partial side elevational views of an exemplary neurovascular stent delivery apparatus 20 configured for selectively positioning and repositioning a stent 22 at a desired location within a blood vessel or another target area within a vascular system of a patient (collectively hereinafter referred to as a “target site” for simplicity purposes), in accordance with at least one embodiment. In at least one embodiment, the stent 22 is comprised of a laser cut tube forming a plurality of interconnecting stent struts 24 cooperating to form a self-expanding stent 22 having an elongated undeployed shape capable of being inserted into a tube such as an introducer sheath or catheter 26 (collectively hereinafter referred to as a “catheter” 26 for simplicity purposes), as best illustrated in FIG. 1. In that regard, it should be noted that the catheter 26 depicted in the drawings is merely exemplary and shown for illustrative purposes. In further embodiments, the apparatus 20 may utilize any type of catheter 26—now known or later developed—capable of allowing the apparatus 20 to substantially carry out the functionality described herein. In at least one embodiment, the stent 22 defines a plurality of stent gaps 28 between the intersecting stent struts 24 along a length of the stent 22. At the outset, it should be noted that the term “strut” is intended in its broadest meaning to include a wire, a fiber, a filament, or other single elongated member.

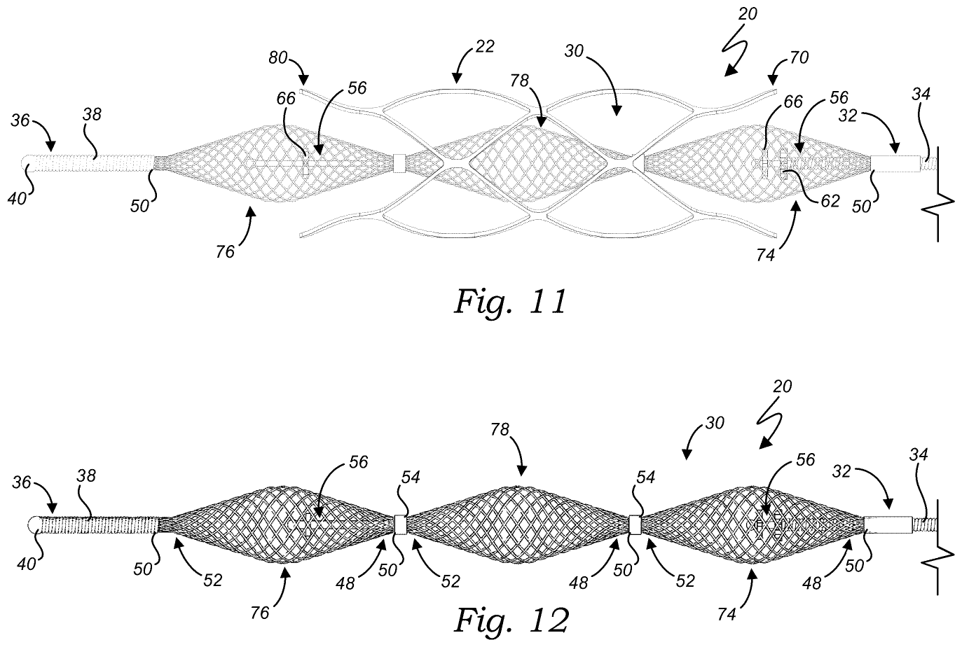

In at least one embodiment, the stent 22 is constructed out of a superelastic metallic material, which allows the stent 22 to automatically expand without externally applied forces. Accordingly, in at least one such embodiment, the stent 22 is configured for changing between one of an elongated undeployed shape—wherein an outer stent diameter of the stent 22 is small enough to allow the stent 22 to be inserted into a catheter 26 (as best illustrated in FIG. 1)—and a relatively larger deployed shape (an example of which is illustrated in FIG. 11)—wherein the outer stent diameter is sized and configured for allowing the stent 22 to effectively fill the target site. In other words, the stent 22 is biased into to its deployed shape, such that as the stent 22 exits the catheter 26 during use of the apparatus 20, the stent 22 automatically moves into its deployed shape. Exemplary deployed shapes of the stent 22 include tubular, helices, vortices, flat spirals, complex spirals, three-dimensional complex shapes, spherical, or any other shape—now known or later developed—capable of effectively filling the target site, as required by any specific clinical application of the apparatus 20. In further embodiments, the stent 22 may be constructed out of any other materials, now known or later developed, capable of biasing the stent 22 into its deployed shape. In at least one alternate embodiment, the stent 22 is configured for being manually moved between its undeployed shape and deployed shape using any mechanisms or techniques now known or later developed.

In at least one embodiment, where the stent 22 is comprised of a plurality of stent struts 24 formed together or otherwise intersecting, the properties of the stent 22 may be selectively tailored to fit the requirements of the medical procedure for which the apparatus 20 is to be used. For example, in at least one embodiment, one or more individual stent struts 24 may be comprised of materials that are different from the other stent struts 24. In at least one such embodiment, at least one of the stent struts 24 is comprised of one or more radiopaque materials (such as a tungsten-loaded polymer, platinum, chromium, cobalt, tantalum, nitinol, gold, silver, bismuth subcarbonate, barium sulfate, bismuth oxychloride, bismuth trioxide, stainless steel or alloys thereof, or any other radiopaque material now known or later developed), while the remaining stent struts 24 are comprised of one or more non-radiopaque materials (such as fiber, plastic, polymers, multi-layer composites or other biocompatible materials, for example). In this way, the radiopacity of the stent 22 may be selectively tailored (by including a lower or higher quantity of radiopaque stent struts 24) while also maintaining the requisite structural integrity for necessary shape retention. In at least one further embodiment, one or more individual stent struts 24 may be comprised of both radiopaque and non-radiopaque materials. In at least one further embodiment, one or more individual stent struts 24 may be comprised of drawn filled tube bi-materials or compounded bimetals. Those skilled in the art will understand that other suitably bio-compatible materials may be used so long as they possess appropriate mechanical properties. It should also be noted that the specific stent strut 24 patterns illustrated in the accompanying figures are merely exemplary and are being shown and described for illustrative purposes only. In further embodiments, the stent struts 24 may take on any other patterns, now known or later developed, so long as the apparatus 20 is able to substantially carry out the functionality described herein—so long as the apparatus 20 is able to substantially carry out the functionality described herein. Similarly, in still further embodiments, where the stent 22 is comprised of a plurality of stent struts 24 braided together or otherwise intersecting, the stent struts 24 may be braided using any braid, knit, or weave patterns now known or later developed (the terms “braid” and “braided” as used herein intended to be all-encompassing for simplicity purposes)—including but in no way limited to 1-over-1-under-1, 1-over-2-under-2, 2-over-2-under-2, etc.—so long as the apparatus 20 is able to substantially carry out the functionality described herein. Additionally, the stent gaps 28 depicted in the drawings are merely exemplary as well. In still further embodiments, the stent gaps 28 may take on any other sizes, shapes, quantities, dimensions or patterns, now known or later developed, so long as the apparatus 20 is able to substantially carry out the functionality described herein. Similarly, the stent struts 24 themselves may each take on any other sizes, quantities or dimensions, now known or later developed, so long as the apparatus 20 is able to substantially carry out the functionality described herein. For example, in at least one embodiment, one or more of the stent struts 24 may have a round shape, a flat shape, a square shape, a hexagonal shape, an oval shape, a ribbon shape, a hollow/tubular shape, etc. in cross-section. Accordingly, in further embodiments, the stent 22 may take on any other sizes, shapes and/or dimensions, now known or later developed—dependent at least in part on the specific context in which the apparatus 20 is to be utilized—so long as the stent 22 is able to substantially carry out the functionality described herein.

In at least one embodiment, the apparatus 20 provides a delivery mechanism 30 configured for being selectively positionable coaxially within and removably engageable with the stent 22, as discussed further below. In at least one embodiment, a proximal end 32 of the delivery mechanism 30 provides or is otherwise engaged with a proximal push wire 34 configured for moving the apparatus 20 through the catheter 26. In at least one embodiment the proximal push wire 34 is a coil or a combination of a wire and a coil to improve deliverability of the delivery mechanism 30 and, in turn, the stent 22 to the target site. However, in further embodiments, the proximal push wire 34 may utilize any other structure, now known or later developed—dependent at least in part on the specific context in which the apparatus 20 is to be utilized—so long as the apparatus 20 is able to substantially carry out the functionality described herein. In at least one embodiment, an opposing distal end 36 of the delivery mechanism 30 provides a distal push wire 38 for assisting in guiding the apparatus 20 through the catheter 26 and to the target site. In at least one embodiment the distal push wire 38 is a coil or a combination of a wire and a coil to improve deliverability of the delivery mechanism 30 and, in turn, the stent 22 to the target site. However, in further embodiments, the distal push wire 38 may utilize any other structure, now known or later developed—dependent at least in part on the specific context in which the apparatus 20 is to be utilized—so long as the apparatus 20 is able to substantially carry out the functionality described herein. In at least one embodiment, the distal push wire 38 terminates to a push wire sphere 40 to create a gentler surface geometry and reduce the risk of damaging the target site during use of the apparatus 20. In at least one embodiment, one or both of the distal push wire 38 and push wire sphere 40 is constructed out of one or more radiopaque materials for improving visibility of the apparatus 20 during use and to facilitate positioning of the apparatus 20 at a desired location relative to the target site.

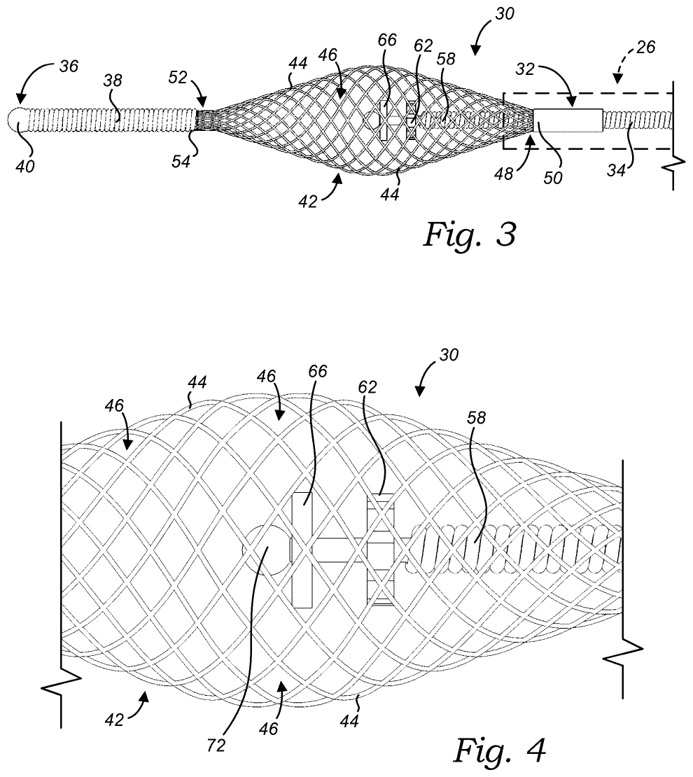

In at least one embodiment, as best illustrated in FIGS. 3 and 4, the delivery mechanism 30 provides an at least one self-expanding delivery sleeve 42 positioned between the proximal push wire 34 and the distal push wire 38. In at least one embodiment, the at least one delivery sleeve 42 is comprised of a plurality of individual sleeve strands 44 braided together or otherwise intersecting to form a self-expanding tubular structure having an elongated undeployed shape capable of being coaxially positioned within the stent 22 when the stent 22 is in its undeployed shape (as best illustrated in FIG. 1). Additionally, the at least one delivery sleeve 42 defines a plurality of sleeve gaps 46 between the intersecting sleeve strands 44 along a length of said delivery sleeve 42.

In at least one embodiment, where the at least one delivery sleeve 42 is comprised of a plurality of sleeve strands 44 braided together, the properties of one or more of the at least one delivery sleeve 42 may be selectively tailored to fit the requirements of the medical procedure for which the apparatus 20 is to be used. For example, in at least one embodiment, one or more individual sleeve strands 44 may be comprised of materials that are different from the other sleeve strands 44. In at least one such embodiment, at least one of the sleeve strands 44 is comprised of one or more radiopaque materials, while the remaining sleeve strands 44 are comprised of one or more non-radiopaque materials. In this way, the radiopacity of the at least one delivery sleeve 42 may be selectively tailored (by including a lower or higher quantity of radiopaque sleeve strands 44) while also maintaining the requisite structural integrity for necessary shape retention. In at least one further embodiment, one or more individual sleeve strands 44 may be comprised of both radiopaque and non-radiopaque materials. In at least one further embodiment, one or more individual sleeve strands 44 may be comprised of drawn filled tube bi-materials or compounded bimetals. Those skilled in the art will understand that other suitably bio-compatible materials may be used so long as they possess appropriate mechanical properties. It should also be noted that the specific braiding patterns illustrated in the accompanying figures are merely exemplary and are being shown and described for illustrative purposes only. In further embodiments, the sleeve strands 44 may be braided using any braid, knit, or weave patterns now known or later developed (the terms “braid” and “braided” as used herein intended to be all-encompassing for simplicity purposes)—including but in no way limited to 1-over-1-under-1, 1-over-2-under-2, 2-over-2-under-2, etc.—so long as the apparatus 20 is able to substantially carry out the functionality described herein. Additionally, braid densities and sleeve gaps 46 depicted in the drawings are merely exemplary as well. In still further embodiments, one or more of the at least one delivery sleeve 42 may utilize any other braid densities, and any sleeve gaps 46 may take on any other sizes, shapes, quantities, dimensions or patterns, now known or later developed, so long as the apparatus 20 is able to substantially carry out the functionality described herein. Similarly, the sleeve strands 44 themselves may each take on any other sizes, quantities or dimensions, now known or later developed, so long as the apparatus 20 is able to substantially carry out the functionality described herein. For example, in at least one embodiment, one or more of the sleeve strands 44 may have a round shape, a flat shape, a square shape, a hexagonal shape, an oval shape, a ribbon shape, a hollow/tubular shape, etc. in cross-section. Accordingly, in further embodiments, one or more of the at least one delivery sleeve 42 may take on any other sizes, shapes and/or dimensions, now known or later developed—dependent at least in part on the specific context in which the apparatus 20 is to be utilized—so long as the at least one delivery sleeve 42 is able to substantially carry out the functionality described herein.

In at least one embodiment, a proximal end 48 of the at least one delivery sleeve 42 provides a proximal end cap 50 for maintaining the braided arrangement of the sleeve strands 44. Similarly, in at least one embodiment, an opposing distal end 52 of the at least one delivery sleeve 42 provides a distal end cap 54 for maintaining the braided arrangement of the sleeve strands 44. In at least one such embodiment, one or both of the proximal end cap 50 and distal end cap 54 is constructed out of one or more radiopaque materials to facilitate positioning of the at least one delivery sleeve 42 at a desired location relative to the target site. In at least one alternate embodiment, one or both of the proximal end 48 and distal end 52 of the at least one delivery sleeve 42 are simply closed, sealed or otherwise finished so as to maintain the braided arrangement of the sleeve strands 44. In at least one embodiment, the proximal end cap 50 is engaged with the proximal push wire 34. In at least one alternate embodiment, the proximal end cap 50 and proximal push wire 34 are one and the same. In at least one embodiment, the distal end cap 54 is engaged with the distal push wire 38. In at least one alternate embodiment, the distal end cap 54 and distal push wire 38 are one and the same.

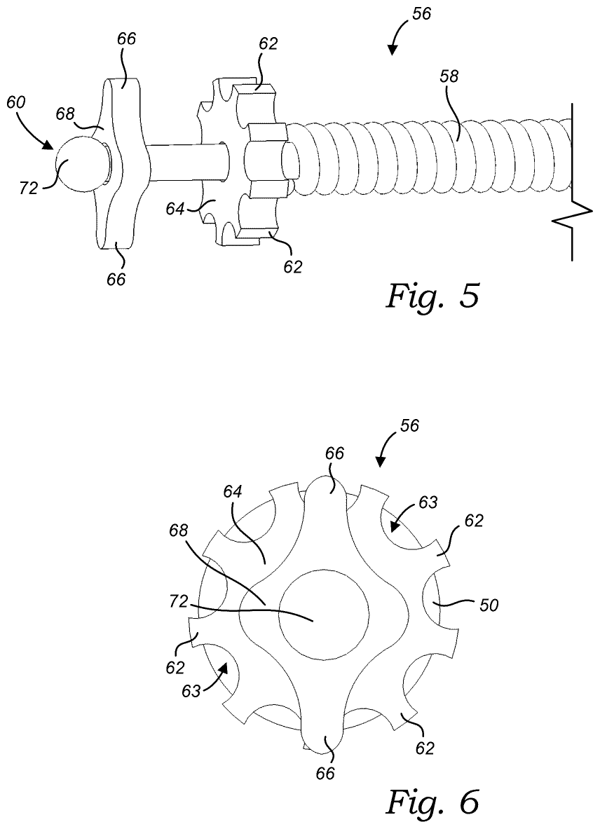

In at least one embodiment, the delivery mechanism 30 further provides an at least one engagement mechanism 56 engaged with the proximal end cap 50 of the at least one delivery sleeve 42 and positioned coaxially within said at least one delivery sleeve 42. In at least one embodiment, as best illustrated in FIGS. 5 and 6, the at least one engagement mechanism 56 provides an elongate engagement shaft 58 engaged with the proximal end cap 50 of the corresponding delivery sleeve 42 and extending a distance toward the distal end cap 54 of said delivery sleeve 42. In at least one embodiment, the engagement shaft 58 lies substantially on a longitudinal centerline of the corresponding delivery sleeve 42. Furthermore, in at least one such embodiment, as best illustrated in FIG. 3, the engagement shaft 58 is substantially linearly aligned with each of the proximal push wire 34 and distal push wire 38. In at least one embodiment, the engagement shaft 58 is a coil or a combination of a wire and a coil to improve deliverability of the delivery mechanism 30 and, in turn, the stent 22 to the target site. However, in further embodiments, the engagement shaft 58 may utilize any other structure, now known or later developed—dependent at least in part on the specific context in which the apparatus 20 is to be utilized—so long as the apparatus 20 is able to substantially carry out the functionality described herein. Additionally, in further embodiments, the engagement shaft 58 may take on any other sizes, shapes, quantities, dimensions and/or relative positions, now known or later developed, so long as the apparatus 20 is able to substantially carry out the functionality described herein.

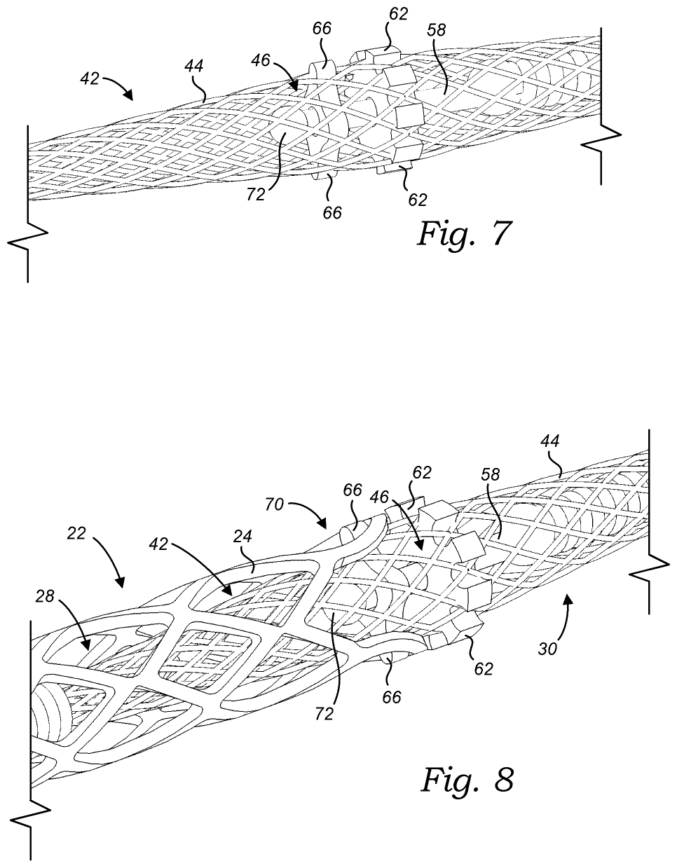

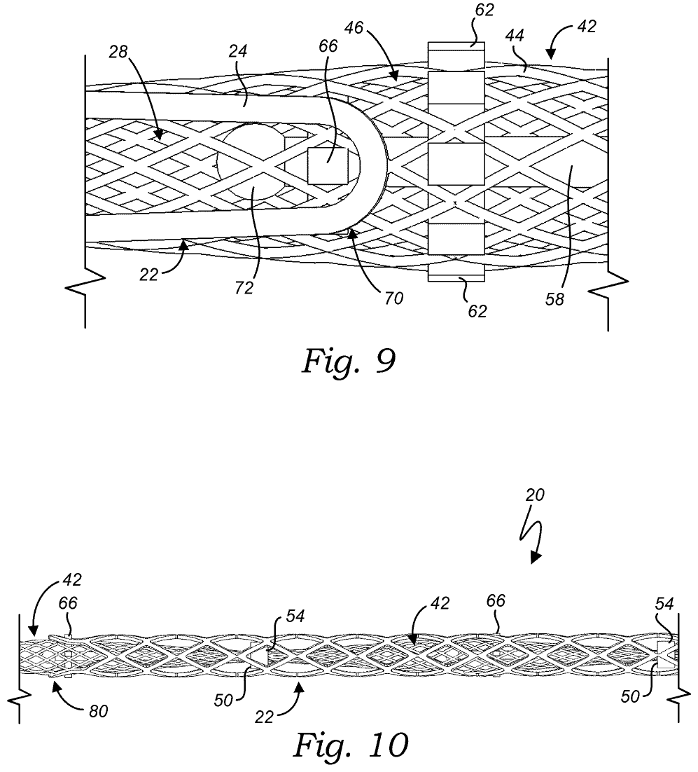

In at least one embodiment, a distal end 60 of the engagement shaft 58 provides an at least one sleeve catch 62 positioned and configured for removable engagement with the corresponding delivery sleeve 42 when the delivery sleeve 42 is in its undeployed shape. In at least one embodiment, the at least one sleeve catch 62 is rotatably engaged with the engagement shaft 58, thereby allowing the at least one sleeve catch 62 to rotate circumferentially about the engagement shaft 58. In at least one alternate embodiment, the at least one sleeve catch 62 is non-rotatably engaged with the engagement shaft 58. In at least one embodiment, the at least one sleeve catch 62 is configured as an elongate protrusion extending radially from the engagement shaft 58, with the at least one sleeve catch 62 being sized and configured for extending through a sleeve gap 46 of the corresponding delivery sleeve 42 when the delivery sleeve 42 is in its undeployed shape, as best illustrated in FIGS. 7 and 9. In at least one embodiment, the engagement shaft 58 provides a plurality of sleeve catches 62 radially spaced apart and arranged about a circumference of the engagement shaft 58, such that the sleeve strands 44 adjacent to the corresponding sleeve gaps 46 sit between the spaced apart sleeve catches 62 when the sleeve catches 62 extend through the sleeve gaps 46. Accordingly, in at least one such embodiment, each pair of adjacent sleeve catches 62 defines a strand notch 63 (FIG. 6) therebetween having a notch width that is relatively greater than a strand diameter of the individual sleeve strand 44 positioned between said adjacent sleeve catches 62, thereby allowing said sleeve strand 44 to sit within the strand notch 63 between said adjacent sleeve catches 62. In at least one such embodiment, the plurality of sleeve catches 62 are provided by a sleeve catch disc 64. It should be noted that the at least one sleeve catch 62 depicted in the drawings is merely exemplary. In further embodiments, the at least one sleeve catch 62 may take on any other sizes, shapes, quantities, dimensions and/or relative positions, now known or later developed, so long as the apparatus 20 is able to substantially carry out the functionality described herein.

In at least one embodiment, the distal end 60 of the engagement shaft 58 further provides an at least one stent catch 66 positioned and configured for removable engagement with the stent 22 when the at least one delivery sleeve 42 and stent 22 are each in their undeployed shapes. In at least one embodiment, the at least one stent catch 66 is rotatably engaged with the engagement shaft 58, thereby allowing the at least one stent catch 66 to rotate circumferentially about the engagement shaft 58. In at least one alternate embodiment, the at least one stent catch 66 is non-rotatably engaged with the engagement shaft 58. In at least one embodiment, the at least one stent catch 66 is configured as an elongate protrusion extending radially from the engagement shaft 58, with the at least one stent catch 66 being sized and configured for extending through both a sleeve gap 46 of the at least one delivery sleeve 42 as well as a stent gap 28 of the stent 22 when the at least one delivery sleeve 42 and stent 22 are each in their undeployed shapes, as best illustrated in FIGS. 8 and 9. In at least one embodiment, the engagement shaft 58 provides a plurality of stent catches 66 radially spaced apart and arranged about a circumference of the engagement shaft 58, such that the stent struts 24 adjacent to the corresponding stent gaps 28, along with the sleeve strands 44 adjacent to the corresponding sleeve gaps 46, sit between the spaced apart stent catches 66 when the stent catches 66 extend through the stent gaps 28 and sleeve gaps 46. Accordingly, in at least one such embodiment, each pair of adjacent stent catches 66 defines a strut notch therebetween having a notch width that is relatively greater than each of a strand diameter of the individual sleeve strand 44 and a strut diameter of the individual stent strut 24 positioned between said adjacent stent catches 66, thereby allowing said sleeve strand 44 and stent strut 24 to sit within the strut notch between said adjacent stent catches 66. In at least one such embodiment, the plurality of stent catches 66 are provided by a stent catch disc 68. It should be noted that the at least one stent catch 66 depicted in the drawings is merely exemplary. In further embodiments, the at least one stent catch 66 may take on any other sizes, shapes, quantities, dimensions and/or relative positions, now known or later developed, so long as the apparatus 20 is able to substantially carry out the functionality described herein.

In at least one embodiment, as best illustrated in FIG. 5, the at least one stent catch 66 is longitudinally spaced apart from the at least one sleeve catch 62 on the engagement shaft 58. In at least one such embodiment, as best illustrated in FIGS. 8 and 9, when the stent 22 is in its undeployed shape, a proximal end 70 of the stent 22 is positioned in the space between the at least one stent catch 66 and the at least one sleeve catch 62. Additionally, in at least one embodiment, as best illustrated in FIG. 6, the at least one stent catch 66 is rotationally offset from the at least one sleeve catch 62 on the engagement shaft 58.

In at least one embodiment, the distal end 60 of the engagement shaft 58 terminates approximately halfway between the proximal end 48 and the distal end 52 of the corresponding delivery sleeve 42. In other words, in at least one embodiment, the engagement shaft 58 has a length that is approximately half the length of the corresponding delivery sleeve 42. However, in at least one alternate embodiment, the engagement shaft 58 could be relatively longer or shorter. In such embodiments, because the engagement shaft 58 is only engaged with the proximal end 48 of the corresponding delivery sleeve 42 and not the distal end 52 as well, each of the at least one sleeve catch 62 and at least one stent catch 66 is capable of floating within the corresponding delivery sleeve 42 while the delivery sleeve 42 is capable of expanding and contracting independent of the at least one sleeve catch 62 and at least one stent catch 66. In at least one alternate embodiment, the engagement shaft 58 of one or more of the at least one delivery sleeve 42 may be attached to the distal end 52 of said delivery sleeve 42 rather than the proximal end 48 of said delivery sleeve 42. In at least one embodiment, the distal end 60 of the engagement shaft 58 terminates to a shaft end 72. In at least one such embodiment, as best illustrated in FIG. 4, the shaft end 72 is substantially spherical shaped so as to create a gentler surface geometry and reduce the risk of damaging the at least one delivery sleeve 42 and stent 22 during use of the apparatus 20. In at least one alternate embodiment, as best illustrated in FIGS. 16 and 17, the shaft end 72 is a bi-petal leaf shape (formed, in at least one such embodiment, by bending a wire approximately 180 degrees). In further alternate embodiments, the shaft end 72 may take on any other sizes, shapes, dimensions and/or configurations, now known or later developed, so long as the apparatus 20 is able to substantially carry out the functionality described herein. In at least one embodiment, one or more of the engagement shaft 58, at least one sleeve catch 62, at least one stent catch 66 and shaft end 72 is constructed out of one or more radiopaque materials for improving visibility of the apparatus 20 during use and to facilitate positioning of the apparatus 20 at a desired location relative to the target site.

In at least one embodiment, similar to the stent struts 24, the sleeve strands 44 of the at least one delivery sleeve 42 are constructed out of a superelastic metallic material, which allows the delivery sleeve 42 to automatically expand without externally applied forces. Accordingly, in at least one such embodiment, the at least one delivery sleeve 42 is configured for changing between one of an elongated undeployed shape—wherein an outer sleeve diameter of the at least one delivery sleeve 42 is small enough to allow the delivery sleeve 42 to be coaxially positioned within the stent 22 when the stent 22 is in its undeployed shape (as best illustrated in FIG. 1)—and a relatively larger deployed shape (an example of which is illustrated in FIG. 3)—wherein the outer sleeve diameter is sized and configured for allowing the delivery sleeve 42 to fully disengage each of the at least one sleeve catch 62 and at least one stent catch 66 of the corresponding engagement mechanism 56 from each of the delivery sleeve 42 and stent 22, as discussed further below. In other words, the at least one delivery sleeve 42 is biased into to its deployed shape, such that as the delivery sleeve 42 exits the catheter 26 during use of the apparatus 20, the delivery sleeve 42 automatically moves into its deployed shape, thereby automatically disengaging each of the at least one sleeve catch 62 and at least one stent catch 66 of the corresponding engagement mechanism 56 from each of the delivery sleeve 42 and stent 22. Exemplary deployed shapes of the at least one delivery sleeve 42 include helices, vortices, flat spirals, complex spirals, three-dimensional complex shapes, spherical, or any other shape—now known or later developed—capable of effectively disengaging each of the at least one sleeve catch 62 and at least one stent catch 66 of the corresponding engagement mechanism 56 from each of the delivery sleeve 42 and stent 22. In further embodiments, the sleeve strands 44 may be constructed out of any other materials, now known or later developed, capable of biasing the at least one delivery sleeve 42 into its deployed shape. In at least one alternate embodiment, the at least one delivery sleeve 42 is configured for being manually moved between its undeployed shape and deployed shape using any mechanisms or techniques now known or later developed.

In at least one embodiment, the outer sleeve diameter of the at least one delivery sleeve 42 is less than an inner stent diameter of the stent 22, thereby allowing the at least one delivery sleeve 42 to be positioned within the stent 22—both in their respective undeployed and deployed shapes. Additionally, in at least one embodiment, at best illustrated in FIG. 4, when the at least one delivery sleeve 42 is in its deployed shape, the outer sleeve diameter of the at least one delivery sleeve 42 has a radius that is greater than a length of each the at least one sleeve catch 62 and at least one stent catch 66, such that when the at least one delivery sleeve 42 moves from its undeployed shape into its deployed shape, each of the at least one sleeve catch 62 and stent catch 66 become disengaged from both the at least one delivery sleeve 42 and the stent 22. Accordingly, during use of the apparatus 20, with each of the at least one delivery sleeve 42 and stent 22 positioned within the catheter 26 in their respective undeployed shapes, upon the catheter 26 reaching the target site, the proximal push wire 34 is used to push the at least one delivery mechanism 30 which, in turn, advances the at least one delivery sleeve 42 and stent 22 (via the corresponding at least one sleeve catch 62 and at least one stent catch 66) through the catheter 26. As the distal ends 52 and 80 of each of the at least one delivery sleeve 42 and stent 22 exit the catheter 26, each of the at least one delivery sleeve 42 and stent 22 begin to automatically change into their respective deployed shapes. Upon the proximal ends 48 and 70 of each of the at least one delivery sleeve 42 and stent 22 exiting the catheter 26, each of the at least one delivery sleeve 42 and stent 22 fully change into their respective deployed shapes, thereby causing each of the at least one sleeve catch 62 and stent catch 66 to become disengaged from both the at least one delivery sleeve 42 and the stent 22. The delivery mechanism 30 may then be withdrawn through the catheter 26 and removed from within the stent 22 by pulling the proximal push wire 34, thereby leaving the stent 22 in position relative to the target site. Furthermore, because the outer sleeve diameter of the at least one delivery sleeve 42 has a radius that is greater than a length of each the at least one sleeve catch 62 and at least one stent catch 66 when the at least one delivery sleeve 42 is in its deployed shape, the at least one delivery sleeve 42 effectively prevents the at least one sleeve catch 62 and at least one stent catch 66 from coming into contact with the stent 22 which, in turn, prevents the delivery mechanism 30 from unintentionally moving the stent 22 out of position as the delivery mechanism 30 is being withdrawn.

In at least one embodiment, as best illustrated in FIG. 3, the delivery mechanism 30 provides a single delivery sleeve 42 positioned between the proximal push wire 34 and the distal push wire 38. In at least one such embodiment, the proximal end cap 50 of the delivery sleeve 42 is engaged with the proximal push wire 34. Additionally, in at least one such embodiment, the distal end cap 54 of the delivery sleeve 42 is engaged with the distal push wire 38. In at least one alternate embodiment, the proximal end cap 50 and proximal push wire 34 are one and the same. In at least one alternate embodiment, the distal end cap 54 and distal push wire 38 are one and the same.

In at least one alternate embodiment, as best illustrated in FIGS. 10-12, the delivery mechanism 30 provides a plurality of delivery sleeves 42 linearly arranged and interconnected in series along a length of the delivery mechanism 30, between the proximal push wire 34 and the distal push wire 38. In at least one such embodiment, one or more of the delivery sleeves 42 has a sleeve length and/or sleeve diameter that is different than the sleeve length and/or sleeve diameter of the other linearly arranged and interconnected delivery sleeves 42. In at least one alternate such embodiment, each of the linearly arranged and interconnected delivery sleeves 42 has substantially the same sleeve length and substantially the same sleeve diameter. In at least one embodiment, the quantity of delivery sleeves 42, along with the dimensions of each such delivery sleeve 42, is dependent at least in part on the length of the stent 22, as well as the specific context in which the apparatus 20 is to be utilized. In at least one such embodiment, the delivery mechanism 30 provides a proximal delivery sleeve 74 positioned at the proximal end 32 of the delivery mechanism 30, and a distal delivery sleeve 76 positioned at the distal end 36 of the delivery mechanism 30. In at least one such embodiment, the proximal end cap 50 of the proximal delivery sleeve 74 is engaged with the proximal push wire 34. Additionally, in at least one such embodiment, the distal end cap 54 of the distal delivery sleeve 76 is engaged with the distal push wire 38. In at least one alternate embodiment, the proximal end cap 50 of the proximal delivery sleeve 74 and the proximal push wire 34 are one and the same. In at least one alternate embodiment, the distal end cap 54 of the distal delivery sleeve 76 and the distal push wire 38 are one and the same. In at least one further embodiment, the delivery mechanism 30 provides an at least one intermediate delivery sleeve 78 positioned between the proximal delivery sleeve 74 and the distal delivery sleeve 76. In at least one embodiment, the delivery sleeves 42 are interconnected in series via the proximal and distal end caps 50 and 54. In at least one embodiment, the distal end cap 54 of a given delivery sleeve 42 is also the proximal end cap 50 of the immediately adjacent delivery sleeve 42. In at least one alternate embodiment, the distal end cap 54 of a given delivery sleeve 42 is engaged with the proximal end cap 50 of the immediately adjacent delivery sleeve 42.

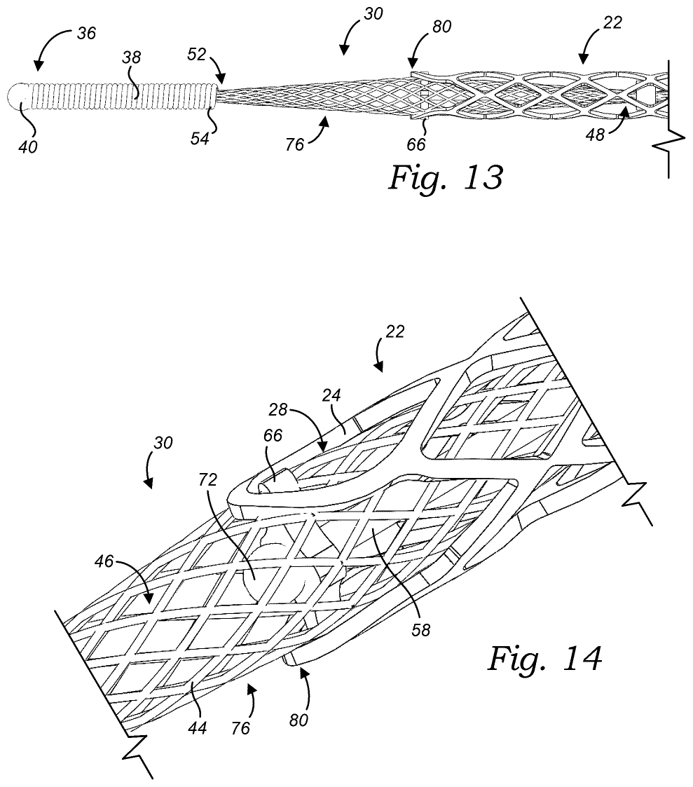

In at least one embodiment, as illustrated in FIGS. 8 and 9, the at least one stent catch 66 of the engagement shaft 58 of the proximal delivery sleeve 74 is configured for removable engagement with the proximal end 70 of the stent 22 when the proximal delivery sleeve 74 and stent 22 are each in their undeployed shapes, such that the at least one stent catch 66 of the engagement shaft 58 of the proximal delivery sleeve 74 is capable of pulling the proximal end 70 of the stent 22 as the proximal push wire 34 is used to pull the at least one delivery mechanism 30 through the catheter 26. Additionally, in at least one embodiment, as illustrated in FIGS. 13 and 14, the at least one stent catch 66 of the engagement shaft 58 of the distal delivery sleeve 76 is configured for removable engagement with a distal end 80 of the stent 22 when the distal delivery sleeve 76 and stent 22 are each in their undeployed shapes, such that the at least one stent catch 66 of the engagement shaft 58 of the distal delivery sleeve 76 is capable of pushing the distal end 80 of the stent 22 as the proximal push wire 34 is used to push the at least one delivery mechanism 30 through the catheter 26. Thus, the at least one stent catch 66 of the engagement shaft 58 of each of the proximal delivery sleeve 74 and distal delivery sleeve 76 cooperate to simultaneously push and pull the respective proximal and distal ends 70 and 80 of the stent 22 through the catheter 26, thereby reducing frictional forces on the stent 22 as the stent 22 moves through the catheter 26. In at least one embodiment, the at least one stent catch 66 of the engagement shaft 58 of each of the at least one intermediate delivery sleeve 78 is configured for removable engagement with the stent 22 in a location between the proximal end 70 and the distal end 80 of the stent 22 when the at least one delivery sleeve 42 and stent 22 are each in their undeployed shapes, thereby further reducing frictional forces on the stent 22 as the stent 22 moves through the catheter 26.

Additionally, as a result, during use of the apparatus 20, as each of the at least one delivery sleeve 42 and stent 22 exit the catheter 26 and begin to automatically change into their respective deployed shapes, each of the at least one sleeve catch 62 and stent catch 66 of each deployed delivery sleeve 42 successively become disengaged from both said delivery sleeves 42 and the stent 22, while each of the at least one sleeve catch 62 and stent catch 66 of each undeployed delivery sleeve 42 still positioned within the catheter 26 remain engaged with said undeployed delivery sleeves 42 and the stent 22. Thus, the at least one sleeve catch 62 and stent catch 66 of each undeployed delivery sleeve 42 within the catheter 26 are capable of continuing to advance said delivery sleeves 42 and stent 22 through the catheter 26 via the proximal push wire 34 until the proximal ends 48 and 70 of each of the at least one delivery sleeve 42 and stent 22 exit the catheter 26, at which point each of the at least one delivery sleeve 42 and stent 22 fully change into their respective deployed shapes, thereby causing each of the at least one sleeve catch 62 and stent catch 66 to become disengaged from both the at least one delivery sleeve 42 and the stent 22.

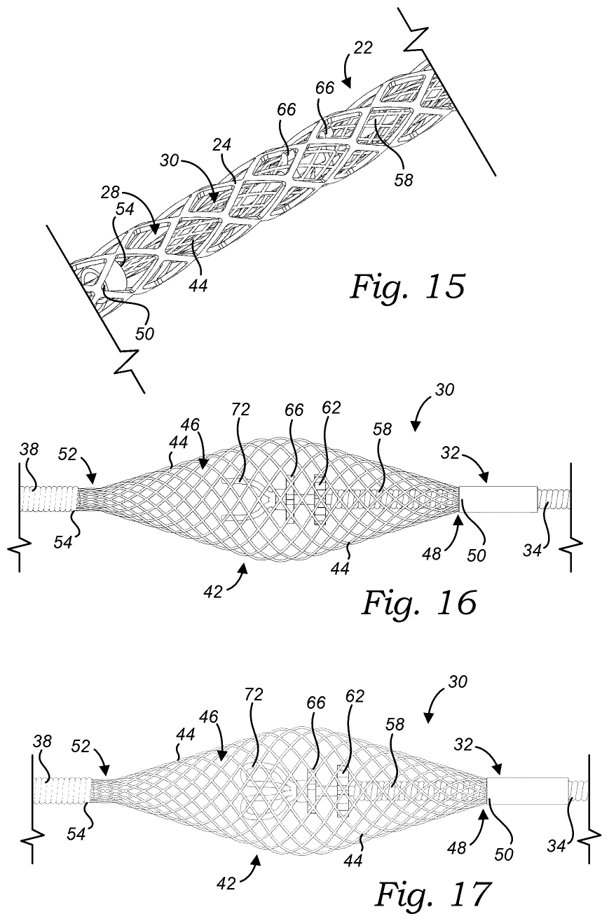

In at least one embodiment, as illustrated in FIG. 15, one or more of the engagement shafts 58 do not provide the at least one sleeve catch 62, such that said engagement shafts 58 only provide the at least one stent catch 66. In at least one such embodiment, the at least one stent catch 66 is configured for both pushing and pulling the stent 22 as described above. In at least one such embodiment, the engagement shaft 58 of the proximal delivery sleeve 74 still provides an at least one sleeve catch 62. Additionally, in at least one embodiment, one or more of the intermediate delivery sleeves 42 do not provide the engagement shaft 58.

Aspects of the present specification may also be described as the following embodiments:
1. A stent delivery apparatus configured for selectively positioning a self-expanding stent relative to a target site, the stent comprising a plurality of intersecting stent struts defining a plurality of stent gaps between the interconnecting stent struts along a length of the stent, the stent configured for moving between one of an elongated undeployed shape—wherein an outer stent diameter of the stent is smaller than an inner catheter diameter of a catheter—and a relatively larger deployed shape—wherein the outer stent diameter is sized and configured for allowing the stent to effectively fill the target site, the apparatus comprising: a delivery mechanism configured for being selectively positionable coaxially within and removably engageable with the stent, the delivery mechanism comprising: a proximal push wire engaged with a proximal end of the delivery mechanism and configured for moving each of the delivery mechanism and the stent through the catheter; a distal push wire positioned at an opposing distal end of the delivery mechanism and configured for assisting in guiding each of the delivery mechanism and the stent through the catheter; and an at least one self-expanding delivery sleeve positioned between the proximal push wire and the distal push wire, each of the at least one delivery sleeve comprising: a plurality of individual sleeve strands braided together so as to define plurality of sleeve gaps between the intersecting sleeve strands along a length of said delivery sleeve, said delivery sleeve configured for moving between one of an elongated undeployed shape—wherein an outer sleeve diameter of said delivery sleeve is smaller than an inner stent diameter of the stent for allowing said delivery sleeve to be coaxially positioned within the stent when the stent is in its undeployed shape—and a relatively larger deployed shape; a proximal end cap positioned at a proximal end of said delivery sleeve for maintaining the braided arrangement of the sleeve strands of said delivery sleeve; a distal end cap positioned at an opposing distal end of said delivery sleeve for maintaining the braided arrangement of the sleeve strands of said delivery sleeve; and an engagement mechanism positioned coaxially within said delivery sleeve, the engagement mechanism providing: an elongate engagement shaft engaged with the proximal end cap and extending a distance toward the distal end cap; and an at least one stent catch positioned substantially on a distal end of the engagement shaft and configured for removable engagement with the stent when said delivery sleeve and the stent are each in their undeployed shapes, the at least one stent catch having a length that is less than a radius of said delivery sleeve when said delivery sleeve is in its deployed shape, such that the at least one stent catch becomes disengaged from said delivery sleeve when said delivery sleeve and the stent are each in their undeployed shapes; whereby, during use of the apparatus, with the at least one delivery sleeve positioned within the stent and the stent positioned within the catheter in their respective undeployed shapes, upon the catheter reaching the target site, the proximal push wire is used to push the delivery mechanism which, in turn, advances the at least one delivery sleeve and stent via the at least one stent catch through the catheter, and upon each of the at least one delivery sleeve and stent exiting the catheter, each of the at least one delivery sleeve and stent automatically change into their respective deployed shapes, thereby causing the at least one stent catch to become disengaged from both the at least one delivery sleeve and the stent.
2. The stent delivery apparatus according to embodiment 1, further comprising the stent.
3. The stent delivery apparatus according to embodiments 1-2, wherein the stent struts are constructed out of a superelastic metallic material, thereby biasing the stent into the deployed shape.
4. The stent delivery apparatus according to embodiments 1-3, wherein at least one of the stent struts of the stent is comprised of an at least one radiopaque material.
5. The stent delivery apparatus according to embodiments 1-4, wherein the distal push wire terminates to a push wire sphere.
6. The stent delivery apparatus according to embodiments 1-5, wherein one or both of the distal push wire and push wire sphere is constructed out of one or more radiopaque materials.
7. The stent delivery apparatus according to embodiments 1-6, wherein at least one of the sleeve strands of the at least one delivery sleeve is comprised of an at least one radiopaque material.
8. The stent delivery apparatus according to embodiments 1-7, wherein the proximal end cap of the at least one delivery sleeve is constructed out of an at least one radiopaque material.
9. The stent delivery apparatus according to embodiments 1-8, wherein the distal end cap of the at least one delivery sleeve is constructed out of an at least one radiopaque material.
10. The stent delivery apparatus according to embodiments 1-9, wherein the proximal end cap of the at least one delivery mechanism is engaged with the proximal push wire.
11. The stent delivery apparatus according to embodiments 1-10, wherein the distal end cap is engaged with the distal push wire.
12. The stent delivery apparatus according to embodiments 1-11, wherein the engagement shaft of the engagement mechanism of the at least one delivery sleeve lies substantially on a longitudinal centerline of said delivery sleeve.
13. The stent delivery apparatus according to embodiments 1-12, wherein the engagement shaft of the engagement mechanism of the at least one delivery sleeve is substantially linearly aligned with each of the proximal push wire and distal push wire.
14. The stent delivery apparatus according to embodiments 1-13, wherein the at least one stent catch is an elongate protrusion extending radially from the engagement shaft of the engagement mechanism of the at least one delivery sleeve, the at least one stent catch sized and configured for extending through both a sleeve gap of said delivery sleeve as well as a substantially linearly aligned stent gap of the stent when said delivery sleeve and the stent are each in their undeployed shapes.
15. The stent delivery apparatus according to embodiments 1-14, wherein the engagement shaft of the engagement mechanism of the at least one delivery sleeve provides a plurality of stent catches radially spaced apart and arranged about a circumference of the engagement shaft.
16. The stent delivery apparatus according to embodiments 1-15, wherein the plurality of stent catches are provided by a stent catch disc.
17. The stent delivery apparatus according to embodiments 1-16, wherein the engagement mechanism of the at least one delivery sleeve further provides an at least one sleeve catch positioned substantially on the distal end of the engagement shaft and configured for removable engagement with said delivery sleeve when said delivery sleeve is in its undeployed shape, the at least one sleeve catch having a length that is less than a radius of said delivery sleeve when said delivery sleeve is in its deployed shape, such that the at least one sleeve catch becomes disengaged from said delivery sleeve when said delivery sleeve is in its deployed shape.
18. The stent delivery apparatus according to embodiments 1-17, wherein the at least one sleeve catch is an elongate protrusion extending radially from the engagement shaft of the engagement mechanism of the at least one delivery sleeve, the at least one sleeve catch sized and configured for extending through a sleeve gap of said delivery sleeve when said delivery sleeve is in its undeployed shape.
19. The stent delivery apparatus according to embodiments 1-18, wherein the engagement shaft of the engagement mechanism of the at least one delivery sleeve provides a plurality of sleeve catches radially spaced apart and arranged about a circumference of the engagement shaft.
20. The stent delivery apparatus according to embodiments 1-19, wherein the plurality of sleeve catches are provided by a sleeve catch disc.
21. The stent delivery apparatus according to embodiments 1-20, wherein the at least one stent catch is longitudinally spaced apart from the at least one sleeve catch on the engagement shaft.
22. The stent delivery apparatus according to embodiments 1-21, wherein, when the stent is in its undeployed shape, a proximal end of the stent is positioned between the at least one stent catch and the at least one sleeve catch.
23. The stent delivery apparatus according to embodiments 1-22, wherein the at least one stent catch is rotationally offset from the at least one sleeve catch on the engagement shaft.
24. The stent delivery apparatus according to embodiments 1-23, wherein the engagement shaft of the engagement mechanism of the at least one delivery sleeve has a length that is approximately half a length of said delivery sleeve.
25. The stent delivery apparatus according to embodiments 1-24, wherein the distal end of the engagement shaft of the engagement mechanism of the at least one delivery sleeve terminates to a shaft end.
26. The stent delivery apparatus according to embodiments 1-25, wherein one or more of the engagement shaft, at least one sleeve catch, at least one stent catch and shaft end is constructed out of one or more radiopaque materials.
27. The stent delivery apparatus according to embodiments 1-26, wherein the delivery mechanism provides a plurality of delivery sleeves linearly arranged and interconnected in series along a length of the delivery mechanism, between the proximal push wire and the distal push wire.
28. The stent delivery apparatus according to embodiments 1-27, wherein the delivery mechanism provides: a proximal delivery sleeve positioned at the proximal end of the delivery mechanism; and a distal delivery sleeve positioned at the distal end of the delivery mechanism.
29. The stent delivery apparatus according to embodiments 1-28, wherein: the proximal end cap of the proximal delivery sleeve is engaged with the proximal push wire; and the distal end cap of the distal delivery sleeve is engaged with the distal push wire.
30. The stent delivery apparatus according to embodiments 1-29, wherein the delivery mechanism provides an at least one intermediate delivery sleeve positioned between the proximal delivery sleeve and the distal delivery sleeve.
31. The stent delivery apparatus according to embodiments 1-30, wherein the delivery sleeves are interconnected in series via the proximal and distal end caps.
32. The stent delivery apparatus according to embodiments 1-31, wherein: the at least one stent catch of the engagement shaft of the proximal delivery sleeve is configured for removable engagement with the proximal end of the stent when the proximal delivery sleeve and stent are each in their undeployed shapes; and the at least one stent catch of the engagement shaft of the distal delivery sleeve is configured for removable engagement with the distal end of the stent when the distal delivery sleeve and stent are each in their undeployed shapes.
33. The stent delivery apparatus according to embodiments 1-32, wherein the at least one stent catch is rotatably engaged with the engagement shaft.
34. The stent delivery apparatus according to embodiments 1-33, wherein the at least one sleeve catch is rotatably engaged with the engagement shaft.
35. The stent delivery apparatus according to embodiments 1-34, wherein each pair of adjacent sleeve catches defines a strand notch therebetween, said strand notch having a notch width that is relatively greater than a strand diameter of the individual sleeve strands of said delivery sleeve, such that the sleeve strands adjacent to the corresponding sleeve gaps sit within the strand notch between said adjacent sleeve catches when said sleeve catches extend through the sleeve gaps of said delivery sleeve when said delivery sleeve is in its undeployed shape.
36. The stent delivery apparatus according to embodiments 1-35, wherein one or more of the delivery sleeves has a sleeve length or a sleeve diameter that is different than a sleeve length or sleeve diameter of the other linearly arranged and interconnected delivery sleeves.
37. The stent delivery apparatus according to embodiments 1-36, wherein each of the linearly arranged and interconnected delivery sleeves has substantially the same sleeve length and substantially the same sleeve diameter.
38. A stent delivery apparatus comprising: a self-expanding stent comprising a plurality of intersecting stent struts defining a plurality of stent gaps between the interconnecting stent struts along a length of the stent, the stent configured for moving between one of an elongated undeployed shape—wherein an outer stent diameter of the stent is small enough to allow the stent to be inserted into a catheter—and a relatively larger deployed shape—wherein the outer stent diameter is sized and configured for allowing the stent to effectively fill the target site; and a delivery mechanism configured for being selectively positionable coaxially within and removably engageable with the stent, the delivery mechanism comprising: a proximal push wire engaged with a proximal end of the delivery mechanism and configured for moving each of the delivery mechanism and the stent through the catheter; a distal push wire positioned at an opposing distal end of the delivery mechanism and configured for assisting in guiding each of the delivery mechanism and the stent through the catheter; and an at least one self-expanding delivery sleeve positioned between the proximal push wire and the distal push wire, each of the at least one delivery sleeve comprising: a plurality of individual sleeve strands braided together so as to define plurality of sleeve gaps between the intersecting sleeve strands along a length of said delivery sleeve, said delivery sleeve configured for moving between one of an elongated undeployed shape—wherein an outer sleeve diameter of said delivery sleeve is smaller than an inner stent diameter of the stent for allowing said delivery sleeve to be coaxially positioned within the stent when the stent is in its undeployed shape—and a relatively larger deployed shape; a proximal end cap positioned at a proximal end of said delivery sleeve for maintaining the braided arrangement of the sleeve strands of said delivery sleeve; a distal end cap positioned at an opposing distal end of said delivery sleeve for maintaining the braided arrangement of the sleeve strands of said delivery sleeve; and an engagement mechanism positioned coaxially within said delivery sleeve, the engagement mechanism providing: an elongate engagement shaft engaged with the proximal end cap and extending a distance toward the distal end cap; and an at least one stent catch positioned substantially on a distal end of the engagement shaft and configured for removable engagement with the stent when said delivery sleeve and the stent are each in their undeployed shapes, the at least one stent catch having a length that is less than a radius of said delivery sleeve when said delivery sleeve is in its deployed shape, such that the at least one stent catch becomes disengaged from said delivery sleeve when said delivery sleeve and the stent are each in their undeployed shapes; whereby, during use of the apparatus, with the at least one delivery sleeve positioned within the stent and the stent positioned within the catheter in their respective undeployed shapes, upon the catheter reaching the target site, the proximal push wire is used to push the delivery mechanism which, in turn, advances the at least one delivery sleeve and stent via the at least one stent catch through the catheter, and upon each of the at least one delivery sleeve and stent exiting the catheter, each of the at least one delivery sleeve and stent automatically change into their respective deployed shapes, thereby causing the at least one stent catch to become disengaged from both the at least one delivery sleeve and the stent.
39. A stent delivery apparatus configured for selectively positioning a self-expanding stent relative to a target site, the stent comprising a plurality of intersecting stent struts defining a plurality of stent gaps between the interconnecting stent struts along a length of the stent, the stent configured for moving between one of an elongated undeployed shape—wherein an outer stent diameter of the stent is smaller than an inner catheter diameter of a catheter—and a relatively larger deployed shape—wherein the outer stent diameter is sized and configured for allowing the stent to effectively fill the target site, the apparatus comprising: a delivery mechanism configured for being selectively positionable coaxially within and removably engageable with the stent, the delivery mechanism comprising: a proximal push wire engaged with a proximal end of the delivery mechanism and configured for moving each of the delivery mechanism and the stent through the catheter; a distal push wire positioned at an opposing distal end of the delivery mechanism and configured for assisting in guiding each of the delivery mechanism and the stent through the catheter; and a plurality of self-expanding delivery sleeves linearly arranged and interconnected in series along a length of the delivery mechanism, between the proximal push wire and the distal push wire, each of the delivery sleeves comprising: a plurality of individual sleeve strands braided together so as to define plurality of sleeve gaps between the intersecting sleeve strands along a length of said delivery sleeve, said delivery sleeve configured for moving between one of an elongated undeployed shape—wherein an outer sleeve diameter of said delivery sleeve is smaller than an inner stent diameter of the stent for allowing said delivery sleeve to be coaxially positioned within the stent when the stent is in its undeployed shape—and a relatively larger deployed shape; a proximal end cap positioned at a proximal end of said delivery sleeve for maintaining the braided arrangement of the sleeve strands of said delivery sleeve; a distal end cap positioned at an opposing distal end of said delivery sleeve for maintaining the braided arrangement of the sleeve strands of said delivery sleeve; and an engagement mechanism positioned coaxially within said delivery sleeve, the engagement mechanism providing: an elongate engagement shaft engaged with the proximal end cap and extending a distance toward the distal end cap; and an at least one stent catch positioned substantially on a distal end of the engagement shaft and configured for removable engagement with the stent when said delivery sleeve and the stent are each in their undeployed shapes, the at least one stent catch having a length that is less than a radius of said delivery sleeve when said delivery sleeve is in its deployed shape, such that the at least one stent catch becomes disengaged from said delivery sleeve when said delivery sleeve and the stent are each in their undeployed shapes; whereby, during use of the apparatus, with the delivery sleeves positioned within the stent and the stent positioned within the catheter in their respective undeployed shapes, upon the catheter reaching the target site, the proximal push wire is used to push the delivery mechanism which, in turn, advances the delivery sleeves and stent via the at least one stent catch through the catheter, and upon each of the delivery sleeves and stent exiting the catheter, each of the delivery sleeves and stent automatically change into their respective deployed shapes, thereby causing the at least one stent catch to become disengaged from both the corresponding delivery sleeve and the stent.

In closing, regarding the exemplary embodiments of the present invention as shown and described herein, it will be appreciated that a neurovascular stent delivery apparatus is disclosed and configured for selectively positioning and repositioning a stent at a desired location relative to a target site. Because the principles of the invention may be practiced in a number of configurations beyond those shown and described, it is to be understood that the invention is not in any way limited by the exemplary embodiments, but is generally directed to a neurovascular stent delivery apparatus and is able to take numerous forms to do so without departing from the spirit and scope of the invention. It will also be appreciated by those skilled in the art that the present invention is not limited to the particular geometries and materials of construction disclosed, but may instead entail other functionally comparable structures or materials, now known or later developed, without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the terms “about” and “approximately.” As used herein, the terms “about” and “approximately” mean that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art, or at least encompassing a range of plus or minus ten percent above and below the value of the stated characteristic, item, quantity, parameter, property, or term.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

Any claims intended to be treated under 35 U.S.C. § 112(f) will begin with the words “means for,” but use of the term “for” in any other context is not intended to invoke treatment under 35 U.S.C. § 112(f). Accordingly, Applicant reserves the right to pursue additional claims after filing this application, in either this application or in a continuing application.

It should be understood that any methods disclosed herein, along with the order in which the respective elements of any such method are performed, are purely exemplary. Depending on the implementation, they may be performed in any order or in parallel, unless indicated otherwise in the present disclosure.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicant and does not constitute any admission as to the correctness of the dates or contents of these documents.

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

What is claimed is:

1. A stent delivery apparatus configured for selectively positioning a self-expanding stent relative to a target site, the stent comprising a plurality of intersecting stent struts defining a plurality of stent gaps between the interconnecting stent struts along a length of the stent, the stent configured for moving between one of an elongated undeployed shape—wherein an outer stent diameter of the stent is smaller than an inner catheter diameter of a catheter—and a relatively larger deployed shape—wherein the outer stent diameter is sized and configured for allowing the stent to effectively fill the target site, the apparatus comprising:
a delivery mechanism configured for being selectively positionable coaxially within and removably engageable with the stent, the delivery mechanism comprising:
a proximal push wire engaged with a proximal end of the delivery mechanism and configured for moving each of the delivery mechanism and the stent through the catheter;
a distal push wire positioned at an opposing distal end of the delivery mechanism and configured for assisting in guiding each of the delivery mechanism and the stent through the catheter; and
an at least one self-expanding delivery sleeve positioned between the proximal push wire and the distal push wire, each of the at least one delivery sleeve comprising:
a plurality of individual sleeve strands braided together so as to define plurality of sleeve gaps between the intersecting sleeve strands along a length of said delivery sleeve, said delivery sleeve configured for moving between one of an elongated undeployed shape—wherein an outer sleeve diameter of said delivery sleeve is smaller than an inner stent diameter of the stent for allowing said delivery sleeve to be coaxially positioned within the stent when the stent is in its undeployed shape—and a relatively larger deployed shape;
a proximal end cap positioned at a proximal end of said delivery sleeve for maintaining the braided arrangement of the sleeve strands of said delivery sleeve;
a distal end cap positioned at an opposing distal end of said delivery sleeve for maintaining the braided arrangement of the sleeve strands of said delivery sleeve; and
an engagement mechanism positioned coaxially within said delivery sleeve, the engagement mechanism providing:
an elongate engagement shaft engaged with the proximal end cap and extending a distance toward the distal end cap; and
an at least one stent catch positioned substantially on a distal end of the engagement shaft and configured for removable engagement with the stent when said delivery sleeve and the stent are each in their undeployed shapes, the at least one stent catch having a length that is less than a radius of said delivery sleeve when said delivery sleeve is in its deployed shape, such that the at least one stent catch becomes disengaged from said delivery sleeve when said delivery sleeve and the stent are each in their undeployed shapes;
whereby, during use of the apparatus, with the at least one delivery sleeve positioned within the stent and the stent positioned within the catheter in their respective undeployed shapes, upon the catheter reaching the target site, the proximal push wire is used to push the delivery mechanism which, in turn, advances the at least one delivery sleeve and stent via the at least one stent catch through the catheter, and upon each of the at least one delivery sleeve and stent exiting the catheter, each of the at least one delivery sleeve and stent automatically change into their respective deployed shapes, thereby causing the at least one stent catch to become disengaged from both the at least one delivery sleeve and the stent.

2. The stent delivery apparatus of claim 1, further comprising the stent.

3. The stent delivery apparatus of claim 1, wherein the proximal end cap of the at least one delivery mechanism is engaged with the proximal push wire.

4. The stent delivery apparatus of claim 1, wherein the distal end cap is engaged with the distal push wire.

5. The stent delivery apparatus of claim 1, wherein the at least one stent catch is an elongate protrusion extending radially from the engagement shaft of the engagement mechanism of the at least one delivery sleeve, the at least one stent catch sized and configured for extending through both a sleeve gap of said delivery sleeve as well as a substantially linearly aligned stent gap of the stent when said delivery sleeve and the stent are each in their undeployed shapes.

6. The stent delivery apparatus of claim 5, wherein the engagement shaft of the engagement mechanism of the at least one delivery sleeve provides a plurality of stent catches radially spaced apart and arranged about a circumference of the engagement shaft.

7. The stent delivery apparatus of claim 1, wherein the engagement mechanism of the at least one delivery sleeve further provides an at least one sleeve catch positioned substantially on the distal end of the engagement shaft and configured for removable engagement with said delivery sleeve when said delivery sleeve is in its undeployed shape, the at least one sleeve catch having a length that is less than a radius of said delivery sleeve when said delivery sleeve is in its deployed shape, such that the at least one sleeve catch becomes disengaged from said delivery sleeve when said delivery sleeve is in its deployed shape.

8. The stent delivery apparatus of claim 7, wherein the at least one sleeve catch is an elongate protrusion extending radially from the engagement shaft of the engagement mechanism of the at least one delivery sleeve, the at least one sleeve catch sized and configured for extending through a sleeve gap of said delivery sleeve when said delivery sleeve is in its undeployed shape.

9. The stent delivery apparatus of claim 8, wherein the engagement shaft of the engagement mechanism of the at least one delivery sleeve provides a plurality of sleeve catches radially spaced apart and arranged about a circumference of the engagement shaft.

10. The stent delivery apparatus of claim 7, wherein the at least one stent catch is longitudinally spaced apart from the at least one sleeve catch on the engagement shaft, such that when the stent is in its undeployed shape, a proximal end of the stent is positioned between the at least one stent catch and the at least one sleeve catch.

11. The stent delivery apparatus of claim 7, wherein the at least one stent catch is rotationally offset from the at least one sleeve catch on the engagement shaft.

12. The stent delivery apparatus of claim 1, wherein the engagement shaft of the engagement mechanism of the at least one delivery sleeve has a length that is approximately half a length of said delivery sleeve.

13. The stent delivery apparatus of claim 1, wherein the delivery mechanism provides a plurality of delivery sleeves linearly arranged and interconnected in series along a length of the delivery mechanism, between the proximal push wire and the distal push wire.

14. The stent delivery apparatus of claim 13, wherein the delivery mechanism provides:
a proximal delivery sleeve positioned at the proximal end of the delivery mechanism; and
a distal delivery sleeve positioned at the distal end of the delivery mechanism.

15. The stent delivery apparatus of claim 14, wherein:
the proximal end cap of the proximal delivery sleeve is engaged with the proximal push wire; and
the distal end cap of the distal delivery sleeve is engaged with the distal push wire.

16. The stent delivery apparatus of claim 14, wherein the delivery mechanism provides an at least one intermediate delivery sleeve positioned between the proximal delivery sleeve and the distal delivery sleeve.

17. The stent delivery apparatus of claim 14, wherein:
the at least one stent catch of the engagement shaft of the proximal delivery sleeve is configured for removable engagement with the proximal end of the stent when the proximal delivery sleeve and stent are each in their undeployed shapes; and
the at least one stent catch of the engagement shaft of the distal delivery sleeve is configured for removable engagement with the distal end of the stent when the distal delivery sleeve and stent are each in their undeployed shapes.

18. The stent delivery apparatus of claim 13, wherein the delivery sleeves are interconnected in series via the proximal and distal end caps.

19. A stent delivery apparatus comprising:
a self-expanding stent comprising a plurality of intersecting stent struts defining a plurality of stent gaps between the interconnecting stent struts along a length of the stent, the stent configured for moving between one of an elongated undeployed shape—wherein an outer stent diameter of the stent is small enough to allow the stent to be inserted into a catheter—and a relatively larger deployed shape—wherein the outer stent diameter is sized and configured for allowing the stent to effectively a target site; and
a delivery mechanism configured for being selectively positionable coaxially within and removably engageable with the stent, the delivery mechanism comprising:
a proximal push wire engaged with a proximal end of the delivery mechanism and configured for moving each of the delivery mechanism and the stent through the catheter;
a distal push wire positioned at an opposing distal end of the delivery mechanism and configured for assisting in guiding each of the delivery mechanism and the stent through the catheter; and
an at least one self-expanding delivery sleeve positioned between the proximal push wire and the distal push wire, each of the at least one delivery sleeve comprising:
a plurality of individual sleeve strands braided together so as to define plurality of sleeve gaps between the intersecting sleeve strands along a length of said delivery sleeve, said delivery sleeve configured for moving between one of an elongated undeployed shape—wherein an outer sleeve diameter of said delivery sleeve is smaller than an inner stent diameter of the stent for allowing said delivery sleeve to be coaxially positioned within the stent when the stent is in its undeployed shape—and a relatively larger deployed shape;
a proximal end cap positioned at a proximal end of said delivery sleeve for maintaining the braided arrangement of the sleeve strands of said delivery sleeve;
a distal end cap positioned at an opposing distal end of said delivery sleeve for maintaining the braided arrangement of the sleeve strands of said delivery sleeve; and
an engagement mechanism positioned coaxially within said delivery sleeve, the engagement mechanism providing:
an elongate engagement shaft engaged with the proximal end cap and extending a distance toward the distal end cap; and
an at least one stent catch positioned substantially on a distal end of the engagement shaft and configured for removable engagement with the stent when said delivery sleeve and the stent are each in their undeployed shapes, the at least one stent catch having a length that is less than a radius of said delivery sleeve when said delivery sleeve is in its deployed shape, such that the at least one stent catch becomes disengaged from said delivery sleeve when said delivery sleeve and the stent are each in their undeployed shapes;
whereby, during use of the apparatus, with the at least one delivery sleeve positioned within the stent and the stent positioned within the catheter in their respective undeployed shapes, upon the catheter reaching the target site, the proximal push wire is used to push the delivery mechanism which, in turn, advances the at least one delivery sleeve and stent via the at least one stent catch through the catheter, and upon each of the at least one delivery sleeve and stent exiting the catheter, each of the at least one delivery sleeve and stent automatically change into their respective deployed shapes, thereby causing the at least one stent catch to become disengaged from both the at least one delivery sleeve and the stent.

20. A stent delivery apparatus configured for selectively positioning a self-expanding stent relative to a target site, the stent comprising a plurality of intersecting stent struts defining a plurality of stent gaps between the interconnecting stent struts along a length of the stent, the stent configured for moving between one of an elongated undeployed shape—wherein an outer stent diameter of the stent is smaller than an inner catheter diameter of a catheter—and a relatively larger deployed shape—wherein the outer stent diameter is sized and configured for allowing the stent to effectively fill the target site, the apparatus comprising:
a delivery mechanism configured for being selectively positionable coaxially within and removably engageable with the stent, the delivery mechanism comprising:
a proximal push wire engaged with a proximal end of the delivery mechanism and configured for moving each of the delivery mechanism and the stent through the catheter;
a distal push wire positioned at an opposing distal end of the delivery mechanism and configured for assisting in guiding each of the delivery mechanism and the stent through the catheter; and
a plurality of self-expanding delivery sleeves linearly arranged and interconnected in series along a length of the delivery mechanism, between the proximal push wire and the distal push wire, each of the delivery sleeves comprising:
a plurality of individual sleeve strands braided together so as to define plurality of sleeve gaps between the intersecting sleeve strands along a length of said delivery sleeve, said delivery sleeve configured for moving between one of an elongated undeployed shape—wherein an outer sleeve diameter of said delivery sleeve is smaller than an inner stent diameter of the stent for allowing said delivery sleeve to be coaxially positioned within the stent when the stent is in its undeployed shape—and a relatively larger deployed shape;
a proximal end cap positioned at a proximal end of said delivery sleeve for maintaining the braided arrangement of the sleeve strands of said delivery sleeve;
a distal end cap positioned at an opposing distal end of said delivery sleeve for maintaining the braided arrangement of the sleeve strands of said delivery sleeve; and
an engagement mechanism positioned coaxially within said delivery sleeve, the engagement mechanism providing:
an elongate engagement shaft engaged with the proximal end cap and extending a distance toward the distal end cap; and
an at least one stent catch positioned substantially on a distal end of the engagement shaft and configured for removable engagement with the stent when said delivery sleeve and the stent are each in their undeployed shapes, the at least one stent catch having a length that is less than a radius of said delivery sleeve when said delivery sleeve is in its deployed shape, such that the at least one stent catch becomes disengaged from said delivery sleeve when said delivery sleeve and the stent are each in their undeployed shapes;
whereby, during use of the apparatus, with the delivery sleeves positioned within the stent and the stent positioned within the catheter in their respective undeployed shapes, upon the catheter reaching the target site, the proximal push wire is used to push the delivery mechanism which, in turn, advances the delivery sleeves and stent via the at least one stent catch through the catheter, and upon each of the delivery sleeves and stent exiting the catheter, each of the delivery sleeves and stent automatically change into their respective deployed shapes, thereby causing the at least one stent catch to become disengaged from both the corresponding delivery sleeve and the stent.
